(19) United States
(12) Patent Application Publication
(10) Pub. No.: US 2025/0032265 A1
(43) Pub. Date: Jan. 30, 2025

(54) Tibial Tray Suture Fixation

(71) Applicant: Arthrex, Inc., Naples, FL (US)

(72) Inventors: Benjamin Chan, Bonita Springs, FL (US); Maris Prieditis, Naples, FL (US); Adam Garlock, Naples, FL (US); Braham Dhillon, Naples, FL (US)

(21) Appl. No.: 18/783,631

(22) Filed: Jul. 25, 2024

Related U.S. Application Data
(60) Provisional application No. 63/515,622, filed on Jul. 26, 2023.

(51) Int. Cl. A61F 2/38; A61F 2/42

(52) U.S. Cl. CPC A61F 2/389; A61F 2002/4205

(57) ABSTRACT

The present disclosure provides prosthetic implants for tibial fixation in a total ankle replacement device. Methods including the devices are also disclosed.

### CROSS-REFERENCE TO RELATED APPLICATIONS

This application claims priority to U.S. Provisional Application No. 63/515,622 entitled “Tibial Tray Suture Fixation,” filed on Jul. 26, 2023, the contents of which are hereby incorporated by reference in its entirety.

### BACKGROUND

Total ankle replacement (TAR) requires fixation of the tibial component to the tibia of a patient, and further requires fixation of the talar component to the talus of the patient. Improving time zero fixation by the inclusion of high retention force features may aide in the long term survival of the prosthetic implant. Accordingly, disclosed herein are prosthetic implants that provide a high retention force to decrease and/or prevent loosening after initial implantation.

### SUMMARY

Total ankle replacement (TAR) requires a prosthesis to replace resected bone and eroded joint cartilage. Existing solutions for the component that replaces the distal tibia leaves room for improvement for initial fixation (time zero fixation) to bone. Some of the challenges lay within the exposure required to use instruments that would adequately prepare the bone for the final implant. Fixation that includes vertical pegs are the most difficult to prepare due to lack of exposure in the ankle joint space. Angled pegs have been used to solve the issue of exposure; however, the time zero fixation for such design leaves room for improvement. Other previous designs have used an anterior approach where the anterior cortex is breached using drills and osteotomes. These drills are large in size and create large bone voids that require back filling with autograft, allograft, or synthetic bone void fillers once the implant is seated. Accordingly, such designs have raised concerns of weakening the weight bearing anterior cortical bone.

The most common failure associated with total ankle replacement (TAR) cases is hardware loosening or subsidence of either the tibial or talar components. Current fixation methods for both the tibial and talar components statically hold the implant in place without providing compression or allowing the implant to dynamize during bone resorption or implant settling.

Accordingly, the present disclosure describes fixation methods for increased fixation during initial implantation between the implant and mating bone. The systems and methods described herein require significantly reduced bone resection for the fixation features of a tibial tray, leaving vastly increased bone stock in case a revision is required. Additionally, hardware removal in the event of a revision may be significantly easier, as suture can be cut and removed easily. The systems and methods described herein also negate the need for large vertical fixation, which may reduce the risk of a commonly known problem of stress shielding.

In one aspect, the present disclosure describes a prosthetic implant including a body comprising a first side and a second side opposite the first side. The first side is configured to contact a tibia of a patient, and the second side is configured to be coupled to an articulation surface. The prosthetic implant further includes at least one fixation component extending away from the first side of the body. The at least one fixation component includes a first end and a second end opposite the first end. The first end of the at least one fixation component includes a button configured to rotate from a first position while traveling through the tibia of the patient to a second position after exiting the tibia of the patient. The second end of the at least one fixation component is coupled to the body. A tension of the at least one fixation component is adjustable to thereby adjust a force applied by the body to the tibia of the patient.

### BRIEF DESCRIPTION OF THE DRAWINGS

FIG. 1 provides an anterior view of an example prosthetic device.

FIG. 2 provides a coronal view of the example prosthetic device of FIG. 1.

FIG. 3 provides a top view and a side view of suture placement of the example prosthetic device of FIG. 1.

FIG. 4 provides a top view of another example prosthetic device.

FIG. 5 provides cross-sectional side view of the example prosthetic device of FIG. 4.

FIG. 6 provides a coronal view of another example prosthetic device.

### DETAILED DESCRIPTION

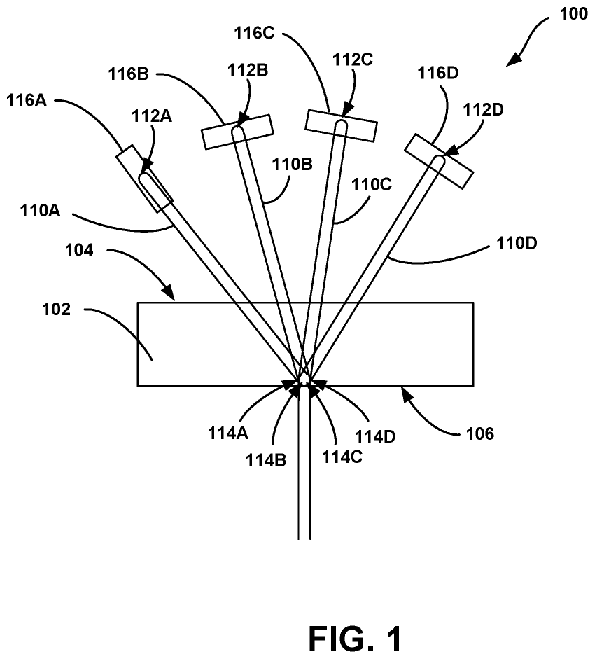

With reference to the Figures, FIGS. 1-5 each illustrate various embodiments of a prosthetic implant configured to be positioned in contact with a tibia of a patient.

As shown in FIGS. 1-5, the prosthetic implant 100 includes a body 102 comprising a first side 104 and a second side 106 opposite the first side 104. The body 102 may comprise a tibial tray as a non-limiting example. The first side 104 is configured to contact a tibia 108 of a patient, and the second side 106 is configured to be coupled to an articulation surface of a total ankle replacement system. The prosthetic implant 100 further includes at least one fixation component 110 extending away from the first side 104 of the body 102. The at least one fixation component 110 includes a first end 112 and a second end 114 opposite the first end 112. The first end 112 of the at least one fixation component 110 includes a button 116 configured to rotate from a first position while traveling through the tibia 108 of the patient to a second position after exiting the tibia 108 of the patient (the first position is shown with button 116A in FIG. 1, and the second position is shown with button 116B in FIG. 1). The second end 114 of the at least one fixation component 110 is coupled to the body 102. A tension of the at least one fixation component 110 is adjustable to thereby adjust a force applied by the body 102 to the tibia 108 of the patient. In one particular example, each of the at least one fixation components 110 are connected to a pair of draw strings that can be used to adjust the tension of the least one fixation component 110. Other arrangements are possible as well.

In an example, the at least one fixation component 110 comprises a suture. In another example, the at least one fixation component 110 comprises a Nitinol wire. In an example, the prosthetic implant 100 further includes one or more pegs 118 coupled to and extending away from the first side 104 of the body 102 to aid in fixation. In one example, the button 116 at the first end 112 of each of the at least one fixation components 110 is a cortical button.

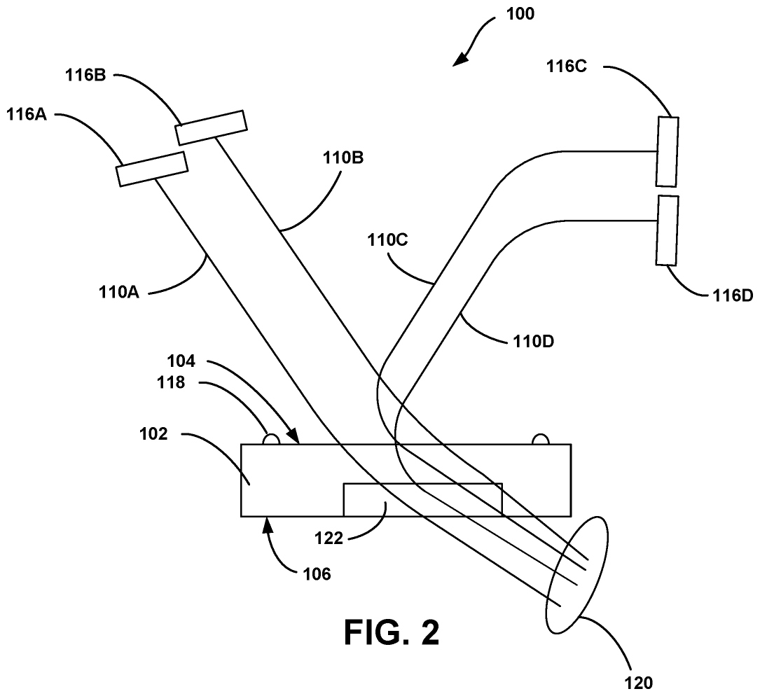

In an example, as shown in FIGS. 1-2, the at least one fixation component 110 comprises a first fixation component 110A, a second fixation component 110B, a third fixation component 110C, and a fourth fixation component 110D. Other numbers of fixation components are possible as well. The prosthetic implant 100 may further include a plate 120 positioned adjacent the second side 106 of the body 102. In such an example, a second end 114A of the first fixation component 110A, a second end 114B of the second fixation component 110B, a second end 114C of the third fixation component 110C, and a second end 114D of the fourth fixation component 110D may each be coupled to the plate 120. In one such example, the second side 106 of the body 102 includes a recess 122 configured to receive the plate 120. Further, the plate 120 may include one or more protrusions configured to mate with one or more recesses in the body 102 to prevent rotation of the plate 120 in a transverse plane. Conversely, the plate 120 may include one or more recesses configured to mate with one or more protrusions of the body 102 to prevent rotation of the plate 120 in a transverse plane.

In one example, a first end 112A of the first fixation component 110A and a first end 112B of the second fixation component 110B are configured to be positioned on an anterior side of the tibia 108 of the patient, and a first end 112C of the third fixation component 110C and a first end 112D of the fourth fixation component 110D are positioned on a posterior side of the tibia 108 of the patient. In an example, the first end 112A of the first fixation component 110A and the first end 112C of the third fixation component 110C are configured to be positioned on a medial side of the tibia 108 of the patient, and the first end 112B of the second fixation component 110B and the first end 112D of the fourth fixation component 110D are positioned on a lateral side of the tibia 108 of the patient.

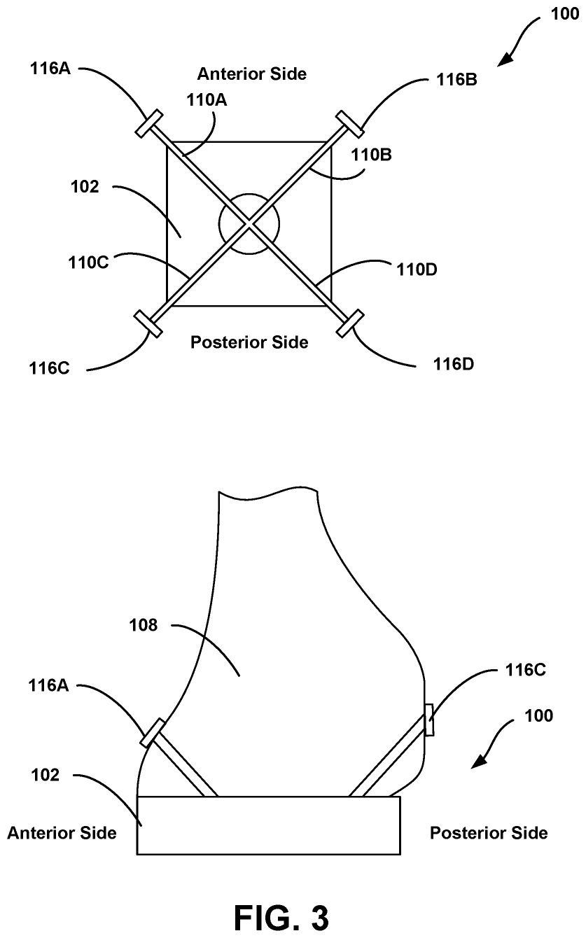

In an example, as shown in FIG. 3, an angle between the first fixation component 110A and the second fixation component 110B in a transverse plane is about 90 degrees, an angle between the second fixation component 110B and the third fixation component 110C in the transverse plane is about 90 degrees, an angle between the third fixation component 110C and the fourth fixation component 110D in the transverse plane is about 90 degrees, and an angle between the fourth fixation component 110D and the first fixation component 110A in the transverse plane is about 90 degrees. The spacing of the fixation components may vary from the arrangement above, but preferably the medial and lateral fixation components and posterior and anterior fixation components should be inserted at equal and opposite angles so that the force vector sums to only exert force in the superior direction.

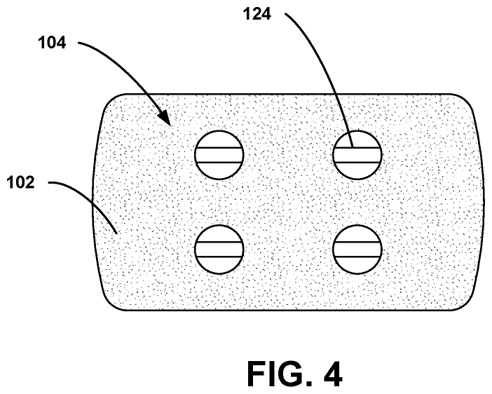

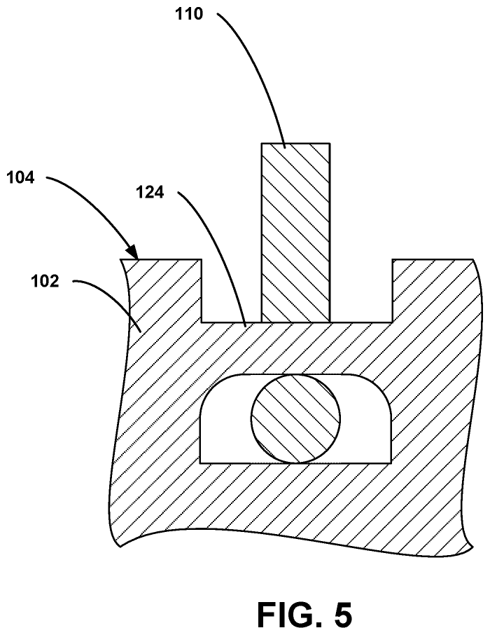

In an example, as shown in FIGS. 4-5, the first side 104 of the body 102 includes one or more dumbbells 124, and the second end 114 of the at least one fixation component 110 is coupled to a respective one of the one or more dumbbells 124. In an example, as shown in FIG. 4, the first side 104 of the body 102 includes a porous surface including four holes with dumbbells 124 positioned therein.

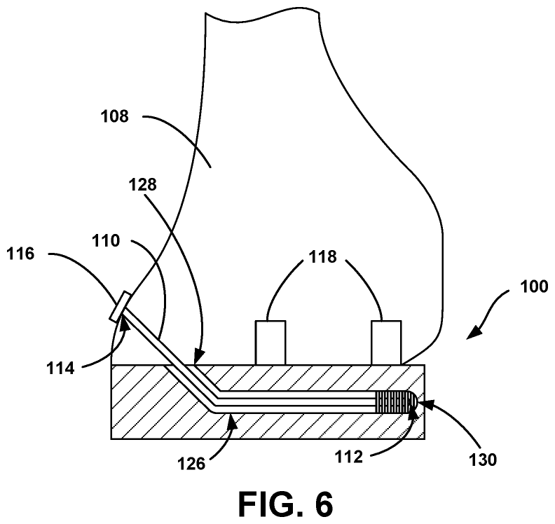

In an example, as shown in FIG. 6, the prosthetic implant 100 further includes a curved channel 126 positioned in the body 102. A first end 128 of the curved channel 126 is open to the first side 104 of the body 102, and a second end 130 of the curved channel 126 terminates in an interior of the body 102. The second end 114 of the at least one fixation component 110 is fixed to the second end 130 of the curved channel 126. The first end 112 of the at least one fixation component 110 extends through the open first end 128 of the curved channel 126 and through the tibia 108 of the patient. In one example, the first end 112 of the at least one fixation component 110 extends through the open first end 128 of the curved channel 126 and through an anterior portion of the tibia 108 of the patient.

Methods disclosed herein can be used with any of the embodiments of the prosthetic implant as described herein.

A method includes prepping all bone resections as per traditional total ankle replacement techniques. Next, a guide surface may be positioned adjacent the tibia 108 of the patient. The guide surface may be disposable, and may have some type of temporary fixation. Next, a targeting guide may be coupled to the guide surface, and anterior holes may be drilled percutaneously through the targeting guide. The targeting guide is removed, and posterior tunnels are drilled blind through the guide surface. This step can be visualized under fluoroscopy if necessary. Next, the button(s) 116 of the at least one fixation component(s) 110 are positioned through the anterior holes using a suture passing device. The suture passing device, such as a nitinol wire as a non-limiting example, pulls the passing sutures, and the button(s) 116 into place. The posterior button(s) 116 may be inserted blind with the use of a rigid insertion device and inserted through the tibia 108. Alternatively, the posterior button(s) 116 may be inserted using a suture passing device similarly to the anterior button(s) 116. Next, the plate 120 is fed thru a hole on the body 102 to the second side 106 of the body 102. The body 102 is positioned as desired. When final positioning is achieved as desired, the tensioner is attached and the sutures are cranked down to a desired tension. The tension can be dialed in precisely as required by the surgeon, and the tension will also set the final features in the body 102 to prevent rotation. Sutures can then be trimmed flush using a standard suture cutter for arthroscopy. An articulation surface (e.g., a poly insert) may be coupled to the second side 106 of the body 102.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and examples have been disclosed herein, other aspects and examples will be apparent to those skilled in the art. The various aspects and examples disclosed herein are for purposes of illustration and are not intended to be limiting, with the true scope being indicated by the following claims, along with the full scope of equivalents to which such claims are entitled. It is also to be understood that the terminology used herein is for the purpose of describing particular examples only, and is not intended to be limiting.

Example methods and systems are described herein. It should be understood that the words “example,” “exemplary,” and “illustrative” are used herein to mean “serving as an example, instance, or illustration.” Any example or feature described herein as being an “example,” being “exemplary,” or being “illustrative” is not necessarily to be construed as preferred or advantageous over other examples or features. The examples described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other examples may include more or less of each element shown in a given Figure. Further, some of the illustrated elements may be combined or omitted. Yet further, an example may include elements that are not illustrated in the Figures.

In the following description, numerous specific details are set forth to provide a thorough understanding of the disclosed concepts, which may be practiced without some or all of these particulars. In other instances, details of known devices and/or processes have been omitted to avoid unnecessarily obscuring the disclosure. While some concepts will be described in conjunction with specific examples, it will be understood that these examples are not intended to be limiting.

As used herein, “coupled” means associated directly as well as indirectly. For example, a member A may be directly associated with a member B, or may be indirectly associated therewith, e.g., via another member C. It will be understood that not all relationships among the various disclosed elements are necessarily represented.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

The limitations of the following claims are not written in means-plus-function format and are not intended to be interpreted based on 35 U. S. C. § 112(f), unless and until such claim limitations expressly use the phrase “means for” followed by a statement of function void of further structure.

By the term “about,” “approximately,” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide. For example, in one embodiment, the term “about” can refer to ±5% of a given value.

Illustrative, non-exhaustive examples, which may or may not be claimed, of the subject matter according the present disclosure are provided below.

What is claimed is:

1. A prosthetic implant comprising:
a body comprising a first side and a second side opposite the first side, wherein the first side is configured to contact a tibia of a patient, and wherein the second side is configured to be coupled to an articulation surface; and
at least one fixation component extending away from the first side of the body, wherein the at least one fixation component includes a first end and a second end opposite the first end, wherein the first end of the at least one fixation component includes a button configured to rotate from a first position while traveling through the tibia of the patient to a second position after exiting the tibia of the patient, wherein the second end of the at least one fixation component is coupled to the body, and wherein a tension of the at least one fixation component is adjustable to thereby adjust a force applied by the body to the tibia of the patient.

2. The prosthetic implant of claim 1, wherein the at least one fixation component comprises a suture.

3. The prosthetic implant of claim 1, wherein the at least one fixation component comprises a Nitinol wire.

4. The prosthetic implant of claim 1, further comprising:
one or more pegs coupled to and extending away from the first side of the body.

5. The prosthetic implant of claim 1, wherein the at least one fixation component comprises:
a first fixation component;
a second fixation component;
a third fixation component; and
a fourth fixation component.

6. The prosthetic implant of claim 5, further comprising:
a plate positioned adjacent the second side of the body, wherein a second end of the first fixation component, a second end of the second fixation component, a second end of the third fixation component, and a second end of the fourth fixation component are each coupled to the plate.

7. The prosthetic implant of claim 6, wherein the second side of the body includes a recess configured to receive the plate.

8. The prosthetic implant of claim 6, wherein the plate includes one or more protrusions configured to mate with one or more recesses in the body to prevent rotation of the plate in a transverse plane.

9. The prosthetic implant of claim 5, wherein a first end of the first fixation component and a first end of the second fixation component are positioned on an anterior side of the tibia of the patient, and wherein a first end of the third fixation component and a first end of the fourth fixation component are positioned on a posterior side of the tibia of the patient.

10. The prosthetic implant of claim 9, wherein the first end of the first fixation component and the first end of the third fixation component are positioned on a medial side of the tibia of the patient, and wherein the first end of the second fixation component and the first end of the fourth fixation component are positioned on a lateral side of the patient.

11. The prosthetic implant of claim 10, wherein an angle between the first fixation component and the second fixation component in a transverse plane is about 90 degrees, wherein an angle between the second fixation component and the third fixation component in the transverse plane is about 90 degrees, wherein an angle between the third fixation component and the fourth fixation component in the transverse plane is about 90 degrees, and wherein an angle between the fourth fixation component and the first fixation component in the transverse plane is about 90 degrees.

12. The prosthetic implant of claim 1, wherein the first side of the body includes one or more dumbbells, and wherein the second end of the at least one fixation component are coupled to the one or more dumbbells.

13. The prosthetic implant of claim 1, further comprising:
a curved channel positioned in the body, wherein a first end of the curved channel is open to the first side of the body, wherein a second end of the curved channel terminates in an interior of the body, wherein the second end of the at least one fixation component is fixed to the second end of the curved channel, wherein the first end of the at least one fixation component extends through the first end of the curved channel and through the tibia of the patient.

14. The prosthetic implant of claim 13, wherein the first end of the at least one fixation component extends through the first end of the curved channel and through an anterior portion of the tibia of the patient.
